SEQUENCE LISTING (11 sequences)

SEQ ID NO: 1 - DNA (cDNA), 4665 bp; single-stranded, linear
  CDS 1..3114
  3′UTR 3115..4665
  polyA-site 4647..4652
agccagcccgaggacgcgagcggcaggtgtgcacagaggttctccactttgttttctgaactcgcggtcaggatggttttctctgtcaggcagtgtggccatgttggcagaactgaagaagttttactgacgttcaagatattccttgtcatcatttgtcttcatgtcgttctggtaacatccctggaagaagatactgataattccagtttgtcaccaccacctgctaaattatctgttgtcagttttgccccctcctccaatgaggttgaaacaacaagcctcaatgatgttactttaagcttactcccttcaaacgaaacagaaaaaactaaaatcactatagtaaaaaccttcaatgcttcaggcgtcaaaccccagagaaatatctgcaatttgtcatctatttgcaatgactcagcattttttagaggtgagatcatgtttcaatatgataaagaaagcactgttccccagaatcaacatataacgaatggcaccttaactggagtcctgtctctaagtgaattaaaacgctcagagctcaacaaaaccctgcaaaccctaagtgagacttactttataatgtgtgctacagcagaggcccaaagcacattaaattgtacattcacaataaaactgaataatacaatgaatgcatgtgctgcaatagccgctttggaaagagtaaagattcgaccaatggaacactgctgctgttctgtcaggataccctgcccttcctccccagaagagttgggaaagcttcagtgtgacctgcaggatcccattgtctgtcttgctgaccatccacgtggcccaccattttcttccagccaatccatcccagtggtgcctcgggccactgtgctttcccaggtccccaaagctacctcttttgctgagcctccagattattcacctgtgacccacaatgttccctctccaataggggagattcaacccctttcaccccagccttcagctcccatagcttccagccctgccattgacatgcccccacagtctgaaacgatctcttcccctatgccccaaacccatgtctccggcaccccacctcctgtgaaagcctcattttcctctcccaccgtgtctgcccctgcgaatgtcaacactaccagcgcacctcctgtccagacagacatcgtcaacaccagcagtatttctgatcttgagaaccaagtgttgcagatggagaaggctctgtccttgggcagcctggagcctaacctcgcaggagaaatgatcaaccaagtcagcagactccttcattccccgcctgacatgctggcccctctggctcaaagattgctgaaagtagtggatgacattggcctacagctgaacttttcaaacacgactataagtctaacctccccttctttggctctggctgtgatcagagtgaatgccagtagtttcaacacaactacctttgtggcccaagaccctgcaaatcttcaggtttctctggaaacccaagctcctgagaacagtattggcacaattactcttccttcatcgctgatgaataatttaccagctcatgacatggagctagcttccagggttcagttcaatttttttgaaacacctgctttgtttcaggatccttccctggagaacctctctctgatcagctacgtcatatcatcgagtgttgcaaacctgaccgtcaggaacttgacaagaaacgtgacagtcacattaaagcacatcaacccgagccaggatgagttaacagtgagatgtgtattttgggacttgggcagaaatggtggcagaggaggctggtcagacaatggctgctctgtcaaagacaggagattgaatgaaaccatctgtacctgtagccatctaacaagcttcggcgttctgctggacctatctaggacatctgtgctgcctgctcaaatgatggctctgacgttcattacatatattggttgtgggctttcatcaatttttctgtcagtgactcttgtaacctacatagcttttgaaaagatccggagggattacccttccaaaatcctcatccagctgtgtgctgctctgcttctgctgaacctggtcttcctcctggactcgtggattgctctgtataagatgcaaggcctctgcatctcagtggctgtatttcttcattattttctcttggtctcattcacatggatgggcctagaagcattccatatgtacctggcccttgtcaaagtatttaatacttacatccgaaaatacatccttaaattctgcattgtcggttggggggtaccagctgtggttgtgaccatcatcctgactatatccccagataactatgggcttggatcctatgggaaattccccaatggttcaccggatgacttctgctggatcaacaacaatgcagtattctacattacggtggtgggatatttctgtgtgatatttttgctgaacgtcagcatgttcattgtggtcctggttcagctctgtcgaattaaaaagaagaagcaactgggagcccagcgaaaaaccagtattcaagacctcaggagtatcgctggccttacatttttactgggaataacttggggctttgccttctttgcctggggaccagttaacgtgaccttcatgtatctgtttgccatctttaataccttacaaggatttttcatattcatcttttactgtgtggccaaagaaaatgtcaggaagcaatggaggcggtatctttgttgtggaaagttacggctggctgaaaattctgactggagtaaaactgctactaatggtttaaagaagcagactgtaaaccaaggagtgtccagctcttcaaattccttacagtcaagcagtaactccactaactccaccacactgctagtgaataatgattgctcagtacacgcaagcgggaatggaaatgcttctacagagaggaatggggtctcttttagtgttcagaatggagatgtgtgccttcacgatttcactggaaaacagcacatgtttaacgagaaggaagattcctgcaatgggaaaggccgtatggctctcagaaggacttcaaagcggggaagcttacactttattgagcaaatgtgattcctttcttctaaaatcaaagcatgatgcttgacagtgtgaaatgtccaattttaccttttacacaatgtgagatgtatgaaaatcaactcattttattctcggcaacatctggagaagcataagctaattaagggcgatgattattattacaagaagaaaccaagacattacaccatggtttttagacatttctgatttggtttcttatctttcattttataagaaggttggttttaaacaatacactaagaatgactcctataaagaaaacaaaaaaaggtagtgaactttcagctaccttttaaagaggctaagttatctttgataacatcatataaagcaactgttgacttcagcctgttggtgagtttagttgtgcatgcctttgttgtatataagctaaattctagtgacccatgtgtcaaaaatcttacttctacatttttttgtatttattttctactgtgtaaatgtattcctttgtagaatcatggttgttttgtctcacgtgataattcagaaaatccttgctcgttccgcaaatcctaaagctccttttggagatgatataggatgtgaaatacagaaacctcagtgaaatcaagaaataatgatcccagccagactgagaaaatgtaagcagacagtgccacagttagctcatacagtgcctttgagcaagttaggaaaagatgcccccactgggcagacacagccctatgggtcatggtttgacaaacagagtgagagaccatattttagccccactcaccctcttnggtgcacgacctgtacagccaaacacagcatccaatatgaatacccatcccctgaccgcatccccagtagtcagattatagaatctgcaccaagatgtttagctttataccttggccacagagagggatgaactgtcatccagaccatgtgtcaggaaaattgtgaacgtagatgaggtacatacactgccgcttctcaaatccccagagcctttaggaacaggagagtagactaggattccttctcttaaaaaggtacatatatatggaaaaaaatcatattgccgttctttaaaaggcaactgcatggtacattgttgattgttatgactggtacactctggcccagccagagctataattgttttttaaatgtgtcttgaagaatgcacagtganaaggggagtagctattgggaacagggaactgtcctacactgctattgttgctacatgtatcgagccttgattgctcctagttatatacagggtctatcttgcttcctacctacatctgcttgagcagtgcctcaagtacatccttattaggaacatttcaaaccccttttagttaagtctttcactaaggttctcttgcatatatttcaagtgaatgttggatctcagactaaccatagtaataatacacatttctgtgagtgctgacttgtctttgcaatatttcttttctgatttatttaattttcttgtatttatatgttaaaatcaaaaatgttaaaatcaatgaaataaatttgcagttaaga

SEQ ID NO: 2 - protein, 1038 aa
SQPEDASGRCAQRFSTLFSELAVRMVFSVRQCGHVGRTEEVLLTFKIFLVIICLHVVLVTSLEEDTDNSSLSPPPAKLSVVSFAPSSNEVETTSLNDVTLSLLPSNETEKTKITIVKTFNASGVKPQRNICNLSSICNDSAFFRGEIMFQYDKESTVPQNQHITNGTLTGVLSLSELKRSELNKTLQTLSETYFIMCATAEAQSTLNCTFTIKLNNTMNACAAIAALERVKIRPMEHCCCSVRIPCPSSPEELGKLQCDLQDPIVCLADHPRGPPFSSSQSIPVVPRATVLSQVPKATSFAEPPDYSPVTHNVPSPIGEIQPLSPQPSAPIASSPAIDMPPQSETISSPMPQTHVSGTPPPVKASFSSPTVSAPANVNTTSAPPVQTDIVNTSSISDLENQVLQMEKALSLGSLEPNLAGEMINQVSRLLHSPPDMLAPLAQRLLKVVDDIGLQLNFSNTTISLTSPSLALAVIRVNASSFNTTTFVAQDPANLQVSLETQAPENSIGTITLPSSLMNNLPAHDMELASRVQFNFFETPALFQDPSLENLSLISYVISSSVANLTVRNLTRNVTVTLKHINPSQDELTVRCVFWDLGRNGGRGGWSDNGCSVKDRRLNETICTCSHLTSFGVLLDLSRTSVLPAQMMALTFITYIGCGLSSIFLSVTLVTYIAFEKIRRDYPSKILIQLCAALLLLNLVFLLDSWIALYKMQGLCISVAVFLHYFLLVSFTWMGLEAFHMYLALVKVFNTYIRKYILKFCIVGWGVPAVVVTIILTISPDNYGLGSYGKFPNGSPDDFCWINNNAVFYITVVGYFCVIFLLNVSMFIVVLVQLCRIKKKKQLGAQRKTSIQDLRSIAGLTFLLGITWGFAFFAWGPVNVTFMYLFAIFNTLQGFFIFIFYCVAKENVRKQWRRYLCCGKLRLAENSDWSKTATNGLKKQTVNQGVSSSSNSLQSSSNSTNSTTLLVNNDCSVHASGNGNASTERNGVSFSVQNGDVCLHDFTGKQHMFNEKEDSCNGKGRMALRRTSKRGSLHFIEQM

SEQ ID NO: 3 - protein, 14 aa
AFFRGEIMFQYDKE

SEQ ID NO: 4 - protein, 15 aa
CLADHPRGPPFSSSQ

SEQ ID NO: 5 - protein, 15 aa
GSYGKFPNGSPDDFC

SEQ ID NO: 6 - protein, 10 aa
CWINNNAVFY

SEQ ID NO: 7 - protein, 15 aa
CRIKKKKQLGAQRKT

SEQ ID NO: 8 - DNA (cDNA), 17 bp; single-stranded, linear
agctatgggagctgaag

SEQ ID NO: 9 - DNA (cDNA), 17 bp; single-stranded, linear
tgtcaatggcagggctg

SEQ ID NO: 10 - DNA (cDNA), 17 bp; single-stranded, linear
catccgaaaatacatcc

SEQ ID NO: 11 - DNA (cDNA), 17 bp; single-stranded, linear
tgaaggcacacatctcc

(19) United States
(12) Patent Application Publication
(10) Pub. No.: US 2009/0018071 A1
(43) Pub. Date: Jan. 15, 2009

(54) EPIDIDYMIS-SPECIFIC RECEPTOR PROTEIN

(76) Inventors: Caroline Osterhoff, Hamburg (DE); Richard Ivell, Hamburg (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 12/122,516

(22) Filed: May 16, 2008

Related U.S. Application Data
(63) Continuation of application No. 10/668,181, filed on Sep. 24, 2003, now abandoned.
Application No. 10/668,181 is a division of application No. 09/731,657, filed on Mar. 12, 2001, now abandoned.
Application No. 09/731,657 is a continuation of application No. 09/041,745, filed on Mar. 13, 1998, now abandoned.

(51) Int. Cl. A61K 38/00; C07K 14/00; C12N 15/11; C12N 15/00; C12N 5/06; C07K 1/00; A61P 15/00; A61K 31/7088; C12N 1/20; C12P 21/04; C07K 16/18

(52) U.S. Cl. 514/12; 530/350; 536/23.5; 435/320.1; 435/325; 435/252.3; 435/69.1; 530/387.9; 514/44; 530/412

(57) ABSTRACT

The present invention relates to a novel transmembrane receptor protein which is specific to the mammalian epididymis, DNA sequences which code for the protein and specific ligands and to the use thereof for the preparation of agents for diagnosis of male infertility and for controlling maturation of spermatozoa.

The invention relates to a novel transmembrane receptor protein which is specific to the mammalian epididymis, DNA sequences which code for the protein and specific ligands, and to the use thereof for the preparation of agents for diagnosis of male infertility and for controlling maturation of spermatozoa.

### BACKGROUND OF THE INVENTION

About 15% of all couples in the Federal Republic of Germany are currently involuntarily childless, and their proportion is increasing constantly. The reasons for the infertility lie equally with the men and women. However, while the reasons in women have been extensively investigated and can be diagnosed, in men organic causes can be established in only approx. 30% of cases. About one third of the remaining 70% of cases can be attributed to oligospermia of unknown origin, while the causes in the remaining cases are still unclear according to the current state of knowledge. In cases of idiopathic infertility in particular, in which no fertilization of the female ovocyte takes place in spite of a sufficient number of spematocca in the ejaculate (H. W. G. Baker et al. (1986) Relative incidence of etiological disorders in male infertility. In: Male Reproductive Dysfunction (ed. R. J. Santen and R. S. Swerdloff), p. 341-372, Marcel Dekker Inc., New York), a disturbance in the maturation of spermatozoa which takes place in the epididymis is suspected.

The epididymis plays a key role in maturation of spermatozoa. In man, it consists of a single tubule about 5 m long, wound up in a meandering form. The still immature spermatozoa formed in the testis are transported to the epididymis, and undergo a maturation process during a passage lasting 2 to 6 days (Moore at al., Fertilizing capacity of rat spermatozoa is correlated with decline in straight-line velocity measured by continuous computer-aided sperm analysis: epididymal rat spermatozoa from the proximal cauda have a greater fertilizing capacity in vitro than those from the distal cauda or vas deferens, Journal of Andrology, 17, 50-60 (1996)). The special medium of the epididymis ensures that the spermatozoa embedded therein remain vital for a long time, and furthermore acquire the particular physical, immunological and biochemical properties of spermatozoa which are capable of fertilization.

It is accordingly to be assumed that in numerous cases male infertility, and in particular idiopathic infertility, is based on disturbances in the maturation processes which take place in the epididymis.

Although the anatomical fine structure of the human epididymis has been described very thoroughly macroscopically, microscopically and by electron microscopy (cf. A. F. Holstein: Morphologische Studien am Nebenhoden des Menschen, Zwanglose Abhandlungan aus dam Geblet der normalen und pathologischen Anatomie (Morphological studies on the epididymis of man, informal papers from the field of normal and pathological anatomyl, 20, 1-91 (1969)), its protein products have been researched only little, with few exceptions (cf., for example, Teton et al., Immunochemical localization of secretory antigens in the human epididymis and their association with spermatozoa, Biology of Reproduction, 32, 591-597 (1985)). Almost all the findings on this organ originate from work on rats, mice, hamsters, boars, bulls or occasionally monkeys, and animal-specific differences are known to be great.

The findings on maturation of spermatozoa during passage through the epididymis obtained on various species of animals can be summarized as follows (cf. T. G. Cooper: The Epididymis, Sperm Maturation and Fertilization, Springer Verlag, Berlin, (1986)):

(a) Development of an orientated forward mobility and capability for hyperactivation of the spermatozoa.

(b) Prevention of premature capacitation, i.e. readiness to perform the acrosome reaction, where decapacitation factors, which are presumably epididymal polypeptides, play a regulating role.

(c) Change in the surface antigens of the spermatozoa in order to promote binding between the spermatozoa and oocyte.

(d) Change in the spermatozoal membrane in order to facilitate fusion with the ovum.

The metabolic event in the epididymis which induces these processes is largely unclear even in the animals studied. However, with the aid of gel electrophoresis it has been shown that some polypeptides are present in the epididymis, in addition to the seminal fluid originating from the rate testis. Among these, the polypeptides (a) to (e) of Cooper which contain so-called “acidic epididymal glycoprotein (AEG)” (Lea et al., (1978)) and polypeptides B to E of Brooks and colleagues (D. E. Brooks and J. Higgins, Characterization and androgen-dependence of proteins associated with luminal fluid and spermatozoa in the rat epididymis, Journal of Reproduction and Fertility 59, 363-375, (1980)) are present in the rat. The results of these models indicate that the formation of these polypeptides is influenced by androgens.

However, the findings obtained from animal studies cannot be applied to humans because of the high tissue and species specificity. For example, the epididymal polypeptides essential in the rat or the mRNAs coding them have not been found in any other tissue and, apart from the mouse, in any other species (D. E. Brooks et al., Europ. J. Biochem., 161, 13-18 (1986); J. Biolog. Chem., 261, 4956-4961, (1986)).

EP-A-0 440 321 discloses 5 specific polypeptides of the human epididymis and nucleotide sequences which code for these polypeptides which are closely connected with maturation of spermatozoa in the epididymis. These nucleotide sequences and the corresponding expression products, as well as antibodies directed against them, are suitable for diagnosis and use in the treatment of male infertility.

### SUMMARY OF THE INVENTION

It is an object of the present invention to provide methods of diagnosis and treatment of male infertility, such as when caused by protein metabolism disturbances in the epididymis and compounds and compositions to perform these methods.

In one embodiment, the present invention provides epididymis-specific receptor protein (ESRP), represented as SEQ ID NO: 2, and active derivatives or protein fragments thereof having the same biological activity and/or immunogenicity, and DNA sequences which code for the ESRP in particular those according to SEQ ID NO: 1 and DNA sequences derived from these, DNA which code for the protein fragments, and, taking into consideration the degeneration of the genetic code, DNA sequences which coincide with these DNA sequences are within the scope of the present invention. (Osteroff, C; Ivell, R, Kirchhoff, C, DNA Cell Biol (1997) April; 16(4):379-389.)

In another embodiment, the present invention provides homologous amino acid sequences and corresponding DNA sequences having a degree of homology or similarity of at least about 700%, preferably at least about 80%, and particularly preferably at least about 90%. Finally, the invention also relates to nucleotide sequences which hybridize with sequences described herein, under the conditions defined below and are suitable, in particular, as probes.

Prior to the present invention, it had not been possible to isolate and identify epididymis-specific receptor proteins participating in production of the epididymis medium described above, by conventional methods, because of the small quantities of tissue available.

It has now been possible, for the first time, to detect and characterize a specific transmembrane receptor protein of the mammalian epididymis and to enable its preparation by means of recombinant processes or chemical synthesis.

To identify the receptor protein according to the invention, a cDNA library of human epididymal mRNA in lambda gt11 (Clontech, California) was first established, and the epididymis-specific recombinants were then selected in a sequence of differential hybridization steps (cf. FIGS. 1 and 3 to 5) and then subcloned in the bacterial plasmid pBS (Stratagene, California, USA) and characterized.

The aim of the differential screening was to isolate clones of mRNAs which are present in the tissue of the epididymis but not in the tissue of other organs and organs of related function. In the process employed according to the invention, human testicular tissue was used as the comparison material in the first step. Since some of the tissue samples used as the starting material originated from orchiectomized men who had been treated with various medicaments, it was first investigated whether and to what extent the medication has an influence on the particular polypeptide expression pattern. However, it was demonstrated on 7 patients of different age and different medication (cf. example 2 and FIG. 2a to c) that the patterns are influenced only insignificantly by prior administration of medicaments, and also that the individual deviations are slight.

As shown in the diagram in FIG. 1, screening was carried out according to the invention in a primary and a secondary stage, potentially epididymis-specific clones being isolated in the primary stage by absence of cross-hybridization with testis mRNA, and these being selected further in the secondary stage by absence of cross-hybridization with mRNA from the brain and liver (cf. example 3 and FIGS. 3 and 4).

By subsequent northern blot analysis against total mRNA from testes and human decidua, the number of possible clones could be concentrated further, and finally assigned to six independent cDNA families (cf. example 3 and FIG. 5). The cDNA clone, according to SEQ ID NO: 1, coding for the receptor protein according to the invention was isolated from one of these cDNA families.

The results obtained according to the invention show that the mRNA on which this clone is based is not synthesized in other human tissues (cf. FIG. 6; it is accordingly an epididymis-specific molecule. FIGS. 6 and 7 show the underlying autoradiograms of Northern blot hybridization in each case.

The nucleotide sequence and the corresponding amino acid sequence of the cDNA clone according to the invention were analysed as described in example 7. The result is shown in SEQ ID NO: 1.

The specificity of this clone and therefore of the corresponding expression product can be seen from its exclusive northern hybridization with RNA extracted from the human epididymis which could not be detected by cross-hybridization in any other human tissue studied, including the organs of the brain and liver. The tissue specificity was furthermore confirmed by the fact that a homologous gene probe produced from the rat using PCR technology also hybridized exclusively with RNA from the epididymal tissue, but not with RNA from another tissue of this animal species (cf. FIG. 7).

Complete sequencing of a series of homologous cDNA clones isolated from two human epididymal cDNA libraries and determined using the 5′ RACE method (M. A. Frohman, RACE: Rapid Amplification of cDNA ends, in: PCR Protocols: A Guide to Methods and Applications, Academic Press Inc., p. 28-38 (1990)) gave a uniform gene transcript according to SEQ ID NO: 1 with a length of 4,665, nucleotides, which corresponds to the length of the mRNA determined by northern hybridization. This sequence comprises a complete reading frame which codes for a polypeptide of 1,088 amino acids (SEQ ID NO; 2).

The homology or similarity of the amino acid sequences and DNA sequences according to the invention. (SEQ ID NO: 1 and 2) with the sequences stored in various international databanks (NIH Genbank database, EMBL, PIR) was analysed. It was found that the maximum homology found was with the “secretin/VIP superfamily” of G protein-coupled receptors (W. C. Probst et al., Sequence Alignment of G-Protein Coupled Receptor Superfamily, DNA and Cell Biology, 11, p. 1-20 (1992)) and was 25%.

The present invention, therefore, provides a hitherto unknown receptor, the long N-terminal end of which indicates an extracellular domain comparable to that of glycoprotein receptors, i.e. a new receptor for a large glycoprotein ligand originating from testicular secretion. With the aid of a hydrophobicity analysis (example 7), seven clearly defined hydrophobic regions were demonstrated, which are to be designated transmembrane domains (cf. FIG. 8a). The structure of the protein according to the invention characterizes this as a membrane-resident receptor which is to be assigned to the gene family of G protein-coupled receptors (see above). It has the G protein binding domains typical of this group of proteins, with the aid of which information is transduced through the receptor and passed on to the inside of cells via G proteins (L. Birnbaumer, J. Abramowitz and A. M. Brown, Receptor-effector coupling by G proteins, Biochem. Biophys. Act., 1031, 163-224 (1990)).

The receptor protein according to the invention is present in the epididymis in a high abundance (the mRNA makes up about 0.01% of the cDNA library) and in situ hybridization studies localized the mRNA in the epithelial cells which line the ductus epididymis (FIG. 9a).

Furthermore, the receptor protein according to the present invention, is a molecule which is highly conserved among mammalian species, since in the mammalian species studied to date, homology of the expression products of about 90% was found, with the same tissue specificity (FIGS. 7 and 9c, d).

The nucleotide sequences which code for the new receptor protein and fragments according to the invention can be transferred into prokaryotic or eukaryotic host cells by conventional methods via suitable vectors and expressed there as protein. The present invention includes vectors and host cells containing these nucleotide sequences as well as host cells transformed by same, and the recombinantly expressed proteins and fragments.

All the DNA sequences which, after transformation of suitable prokaryotic and/or eukaryotic host cells, ensure the production of nucleic acids for use as diagnostics and/or the expression of proteins or polypeptides which have at least a portion of the primary structure and one or more of the biological and/or immunogenic properties of the receptor protein according to the invention are suitable and included according to the invention. These sequences, in single- or double-stranded form, include, in particular:

a) The nucleotide sequence represented as SEQ ID NO: 1, the sequence of nucleotides 1 to 3,114 of SEQ ID NO: 1, nucleoside, sequences which are homologous to the above-mentioned sequences with a degree of homology or similarity of at least about 70%, and fragments thereof, where these fragments code for polypeptides or proteins having the same biological activity and/or immunogenicity, and syngenic or complementary sequences;

b) nucleotide sequences which hybridize with the protein-coding region of the nucleotide sequence described under a), for example under the hybridization conditions described in examples 3 or 4;

c) nucleotide sequences which, with the exception of the deviations caused by the degeneration of the genetic code, hybridize with the sequences mentioned under a; and/or b; and

d) nucleotide sequences which code for the amino acid sequence represented as SEQ ID NO: 2, sequences which are at least about 70% similar, at least about 80% similar, or at least about 90% similar to nucleotide sequences coding for the amino acid sequence represented as SEQ ID NO: 2, and fragments thereof which code for polypeptides or proteins which have at least one biological activity and/or immunogenicity as the protein represented by SEQ ID NO: 2, and syngenic or complementary sequences.

The term “syngenic sequence” includes all sequences which are derived from the same or a homologous or a similar gene and code for the receptor protein in the context of the invention, or can be used for the preparation of probes. The term also includes, in particular, sequences which show deviations on the basis of degeneration of the genetic code, as well as RNA sequences.

Examples of suitable modifications of the DNA sequence are nucleotide substitutions which do not give rise to another amino acid sequence of the ESRP, but which may correspond to the codon usage of the host organism into which the DNA construct is introduced or nucleotide substitutions which do give rise to a different amino acid sequence and therefore, possibly, a different protein structure without, however, impairing the properties of the native variant. Other examples of possible modifications are insertion of one or several nucleotides into the sequence, addition of one or several nucleotides at either end of the sequence, or deletion of one or several nucleotides at either and or within the sequence.

The invention also relates to derivatives in the form of naturally occurring allelic variations of the receptor protein according to the invention or fragments of such allele variations, it being possible for the various allelic forms to differ in respect of sequence length and in respect of deletions, substitutions, insertions or additions of amino acids, both in each case from one another and from the amino acid sequence described in SEQ ID NO: 2.

According to the invention, the term “proteins or polypeptides having the same biological activity and/or immunogenicity” designates molecules which have a) the same epididymal specificity and b) the same ligand-binding capacity as the proteins and polypeptides identified according to the invention. According to the invention, the term “ligand” includes both antibodies against any desired epitopes included in the proteins or polypeptides according to the invention, and other chemical substances or molecules which are capable of binding to one or more of the domains present in the proteins or polypeptides according to the invention. In one embodiment, the present invention provides isolated or purified peptides N1, N2, A, B and C as described in Table I.

Suitable vectors for prokaryotic host cells according to the present invention are, for example, plasmids of the pET series (Rosenburg et al., Vectors for selective expression of cloned DNAs by T7 RNA Polymerase, Gene, 56, p. 125-135 (1987)), of the pGEX series Pharmacia, Freiburg), of the pRIT series (Pharmacia, Freiburg) and of the pH6EX3 series (Berthold et al., Purification of Recombinant Antigenic Epitopes of the Human 68-kDa (U1) Ribonucleoprotein Antigen Using the Expression System pH6EX3 Followed by Metal Chelating Affinity Chromatography, Protein Expression and Purification, 3, p. 50-56 (1992)). Examples of prokaryotic host cells according to the present invention include laboratory strains of Escherichia coli K12 and the strains BL21 DE3) and LE392.

Examples of suitable vector systems according to the present invention for eukaryotic host cells in the case of mammalian cells, are SV40 viruses, (P. W. J. Rigby, Expression of cloned genes in eukaryotic cells using vector systems derived from viral replicons, Genetic Engineering. vol. 3, p. 84-141 (1982)) and vaccinia viruses (Mackett et al., The construction and characterisation of Vaccinia Virus recombinants expressing foreign genes, DNA Cloning, vol. II. (1985), IRL Press, Oxford), and derivatives thereof, as well as plasmids which contain parts of viral genes (e.g. pSV2) and can be employed as “shuttle vectors” (P. W. J. Rigby). Possible vectors for insect cells are, for example, pJVETL baculoviruses (Invitrogen, San Diego, Calif., USA), while for yeast cells, e.g., pJP31 and YEp- and YIp-plasmids (Carter et al., Expression and secretion foreign genes in yeast. DNA Cloning, vol. III, (1987), IRL Press, Oxford) can be used.

Suitable mammalian cells are e.g. COS cells, CHO cells, AtT20 cells, NIH3T3 cells (Rigby, Mackett et al., HEK293 cells (American Type Cell Collection (ATCC) Nc. CRL 1573) and MDCK cells (European Collection of Animal Cell Cultures No. 85011435), while suitable insect cells are e.g. Sf9, Sf21 and TN5 (Invitrogen, San Diego, Calif., USA). Suitable yeast cells are e.g. X4003-5B (Carter).

The receptor protein according to the invention can furthermore be synthesized chemically by known processes (J. M. Stewart, Synthesis and use of neuropeptides, Neuroendocrine Peptide Methodology, ed. P. M. Conn, Academic Press, NewYork, p. 815-844 (1989)). The same applies to polypeptides or peptide epitopes having the same immunogenicity which are coded by fragments or syngenic sequences of the DNA sequence according to the invention (SEQ ID NO: 1). This is also true for the oligopeptides N1 (SEQ ID NO: 3), N2 (SEQ ID NO: 4), A (SEQ ID NO: 5), B (SEQ ID NO: 6) and C (SEQ ID NO: 7) (table I).

The polypeptides and proteins according to the invention, the nucleotide sequences which code for these, including their complementary sequences, and antibodies produced on the basis of the polypeptides and proteins offer for the first time the possibility of diagnosing and, where appropriate, treating disturbances in the protein metabolism of the epididymal epithelium, and of providing new contraceptive agents.

Thus, for example, the above mentioned nucleotide sequences of the present invention can be provided with markers and used as probes for in situ hybridization in tissue diagnostics of biopsy samples or thin sections, in order to determine the physiological state of the tissue in respect of the presence and concentration of the receptor proteins according to the invention, and to compare it with standard values.

Polyclonal and monoclonal antibodies for use in immunological detection methods can be produced in a known manner with the aid of the highly pure polypeptide according to the invention. Such antibodies can be produced on the basis of the complete receptor protein and on the basis of fragments and active derivatives thereof, where these have the same immunogenicity (cf. examples 9).

The antibodies can be marked or labeled and used in vitro or in vivo for detection of the receptor protein according to the invention.

The receptor protein according to the invention and biologically active derivatives or fragments thereof having the same immunogenicity can furthermore be used in marked or labeled or non-marked or unlabeled form as antigens for identification of autoantibodies in the sera of infertile men. This possibility is of particular importance, since it is assumed that in a large proportion of cases infertility is to be attributed to the presence of autoantibodies against essential components of the reproductive system. However, the test methods available to data measure only antibodies directed against some sperm surface antigens, and a sufficiently high titre of the antibodies must be present to allow sperm agglutination to take place. It is assumed, however, that antibodies are present in far lower titres and can cause infertility. These can be detected as an antigen with the purified or isolated receptor protein prepared according to the invention.

Starting from the amino acid sequence disclosed according to the invention, two different processes are available for isolation of antigens for the production of antibodies.

Firstly, a potentially immunogenic region of the protein sequence of interest, which on the one hand is relatively hydrophilic and therefore lies on the outside of the protein molecule, but on the other hand is not impaired in its steric conformation by formation of cysteine disulfide bridges or possible glycosylation sites, can be selected with the aid of a computer.

This peptide region is then synthesized, if appropriate together with flanking amino acids, subsequently coupled to carrier substance and employed as an immunogen for the induction of antibodies (cf. example 9).

Alternatively, for example, the nucleotide sequence which codes for the polypeptide of interest can be cloned into a suitable expression plasmid vector. After subsequent transformation into suitable bacteria, these vectors allow an inducible expression of the coded polypeptide. The bacteriogenic protein or protein fragment prepared in this manner can be used directly, after purification from the bacterial extract, as an antigen for immunization for induction of antibodies (cf. example 8).

The antibodies can by provided with a detectable marker, such as e.g. a fluorescent molecule (fluorophor) and can be used, for example in tissue samples to determine the presence of the epididymis-specific receptor protein in the epididymal epithelium with the aid of immunofluorescence.

The identification and characterization of the receptor protein according to the invention as a highly specific mediator molecule which is present exclusively on the cells of the epididymal epithelium of mammals and is capable of transmitting information for control of the cell function within the cells of the epithelium make it an extremely interesting candidate for diagnosis and, if appropriate, influencing of the abovementioned physiology of the epididymal epithelium. The present invention provides therefore, a therapeutic method and composition to improve the spermatozoa maturation process in the epididymis, for example by administration of the ligand lacking or formed in an inadequate amount in the individual to be treated, in a pharmaceutically acceptable carrier or diluent.

The present invention also provides a contraceptive method and compositions which have a negative influence on the epithelium which can lead to deterioration of spermatozoa maturation. A synthetic ligand which indeed binds firmly to the receptor protein according to the invention but induces no signal transfer or transmission is suitable, for example, for this purpose. Antibodies directed against the receptor protein according to the invention or active derivatives or fragments thereof having the same immunogenicity can furthermore by employed in order to impede binding of and therefore signal inducement by the ligand or ligands specific to the receptor protein by way of competitive displacement and are also a part of the present invention.

Using certain methods, such as e.g. phage display and peptide display (J. K. Scott and G. P. Smith, Searching for peptide ligand with an epitope library, Science 249, p. 386-390 (1990)); J. J. Devlin et al., Random peptide libraries: a source of specific protein binding molecules, Science 249, p. 404-406 (1990)), Evolutive Biotechnology (M. Eigen, Selforganization of matter and the evolution of biological macromolecules, Die Naturwissenschaften 58, p. 465-523 (1971); M. Eigen, Automated molecular evolution, Max-Planck Institute of Biophysical Chemistry (1991), one is able to identify synthetic ligands which have the ability to bind specifically and with a high affinity to such a receptor protein and to act either agonistically or antagonistically on the signal transfer capability thereof (ex. 11). Molecules by means of which the physiology of cells which express the receptor protein can be influenced positively (therapeutically) or negatively (contraceptively) can be provided by this route.

Alternatively, the ESRP of the invention, expressed in a host cell, as described herein, may be expressed to retain the transmembrane and, optionally, the cytoplasmic region of the native variant, to be anchored in the membrane of the host cell, and the cells carrying the ESRP may be used as such in the screening or diagnostic assay. Alternatively, the receptor may be a component of membrane preparations, e.g. in solubilised and/or reconstituted form.

The ESRP, derivative or analogue of the invention may be immobilized on a solid support and may, as such, be used as a reagent in the screening methods of the invention. The ESRP, derivative or analogue may be used in membrane-bound form, i.e. bound to whole cells or as a component of membrane preparations immobilised on a solid support.

The solid support employed in the screening methods of the invention preferably comprises a polymer. The support may in itself be composed of the polymer or may be composed of a matrix coated with the polymer. The matrix may be of any suitable material such as glass, paper or plastic. The polymer may be selected from the group consisting of a plastic (e.g. latex, a polystyrene, polyvinylchloride, polyurethane, polyacrylamide, polyvinylalcohol, nylon, polyvinylacetate, and any suitable copolymer thereof, cellulose (e.g. various types of paper, such as nitrocellulose paper and the like), a silicon polymer (e.g. siloxane), a polysaccharide (e.g. agarose or dextran), an ion exchange resin (e.g. conventional anion or cation exchange resins), a polypeptide such as polylysine, or a ceramic material such as glass (e.g. controlled pore glass).

The physical shape of the solid support is not critical, although some shapes may be more convenient than others for the present purpose. Thus, the solid support may be in the shape of a plate, e.g. a thin layer or microtiter plate, or a film, strip, membrane (e.g. a nylon membrane or a cellulose filter) or solid particles (e.g. latex beads or dextran or agarose beads).

It is furthermore contemplated to locate the ligand-binding site on the ESRP of the invention, for instance by preparing deletion or substitution derivatives of the native ESRP (as described herein) and incubating these with ligands known to bind the full-length ESRP and detecting any binding of the ligand to the ESRP deletion derivative. Once the ligand-binding site has been located, this may be used to acquire further information about the three-dimensional structure of the ligand-binding site. Such three-dimensional structures may, for instance, be established by means of protein engineering, computer modelling, NMR technology and/or crystallographic techniques. Based on the three-dimensional structure of the ligand-binding site, it may be possible to design substances which are agonists or antagonist to the ESRP molecule.

The present invention is further explained in detail below with the aid of examples, figures and sequence or protocols.

### BRIEF DESCRIPTION OF THE FIGURES

FIG. 1

Schematic representation of the a) primary and b) secondary screening for the receptor protein according to the invention.

FIG. 2

Two-dimensional electrophoresis patterns of translation products derived from epididymis- or testis-specific poly(A). RNA and synthesized in a cell-free system in the presence of 358-methionine.

a. epididymal tissue of a 74 year-old orchiectomy patient treated with cyproterone acetate;

b. epididymal tissue of a 58 year-old orchiectomy patient not treated with medicaments;

c. testis tissue of the same patient not treated with medicaments (see FIG. 2b).

FIG. 3

Autoradiograms of two replica filters from one of the Petri dishes which were hybridized during the primary screening with

a. complex epididymal cDNA probes or

b. complex testicular cDNA probes.

FIG. 4

Autoradiograms of three replica filters from one of the Petri dishes which were hybridized with complex epididymal cDNA probes, complex liver cDNA probes or complex brain cDNA probes during the secondary screening. ESPR-positive clone is indicated coding for the receptor protein of the invention.

FIG. 5

Examples of autoradiograms of the northern blot analyses of potentially positive cDNA clones, E=epididymis, T=testis, D=decidua

a. Positive cDNA clone, indicated by epididymis specific hybridization;

b. Positive but not epididymis-specific hybridization pattern;

c. Positive cDNA clone (*), which also hybridizes with rRNA

d. cDNA clone without tissue-specific expression.

FIG. 6

Northern blot analysis of human tissues. Blots carrying 20 :g of total RNA per lane [except poly(A). epididymis, and vas deferens lanes which only contained 10 :g of RNA] of various tissues were hybridized with different fragments of the receptor protein of the invention (compare FIG. 8b, bold lines).

a. 1.4-kb 3′UTR

b. 0.7-kb 5′Bam HI

c. 1-kb 5′RACE.

Probes were prepared by random primer extension using [″³²P]dCTP (Sambrook et al., “Molecular Cloning—A Laboratory Manual”, 2nd edition, Cold spring Harbor Laboratory Press (1989)).

FIG. 7

Northern blot analysis of at tissues. Blots carrying 20 :g of total RNA per lane (except for lane 2 containing only 4 :g of RNA) of different rat tissues were hybridized with the E-F1 fragment of the rat homologue of the receptor protein (compare FIG. 8b, PCR subclones). The homologous probe was prepared by random primer extension using [″³²P]dCTP (upper and lower panels). The upper blot was subsequently hybridized to an actin probe to show equal loading (middle panel).

FIG. 8

Diagramatic Representation of Receptor Protein cDNA

a. Hydrophilicity plot of the coding region containing a hydrophobic signal peptide (amino acids 25-62) and seven hydrophobic amino acid stretches between amino acids 642-915 (numbers according to SEQ ID NO: 2).

b. Putative structure of the whole known sequence of the receptor protein of the invention and description of isolated cDNA clones. The predicted coding region (1,038 amino acids) is given as a box, the shaded bars standing for the seven transmembrane domains (I-VII) corresponding to the hydrophilicity plot given in a.

3′ UTR, originally isolated clone from the first 8gt11 epididymal library (library 1);

fragment “virtually complete in length”, clones isolated from the 8 Uni-ZAP library (library 2);

5′ Bam HI and 3′ Kpn I, restriction fragment subclones of the 5′ and 3′ regions;

PCR subclones are the 5′ RACE fragments derived from nested primers (A1, A2) from, the 5′ region of the 3.7-kb cDNA fragment “virtually complete in length” using total human epididymal cDNA, and those derived from primer pair E and F1 using epididymal cDNA from human, rat, mouse, and dog. Bold lines indicate fragments used as probes in other figures.

(A)ₙ,poly(A) tail; C, carboxyl terminus; kb, kilobases; N, amino terminus; nt, nucleotides; SP, putative signal peptide.

c. Structure of Receptors with Seven Helices

(a) Linear diagram of the receptors protein. Seven blocks of hydrophobic amino acids (1-7) are shown. Each assume the shape of a helix, which presumably extends through the membrane. The helices are connected by loops which lie alternately outside (E) and inside (C) the cell. The N-terminus of the receptors points towards the outside, while the C-terminus points inside the cell.

(b) Model of the Assumed Structure of the Folded Receptor.

FIG. 9

In situ localization of receptor protein transcripts in the human and rat epididymis.

a human caput region

b. human caput region

c. rat epididymis (proximal)

d. rat epididymis (distal)

Tissue cryosections were hybridized either with ³⁵S-labeled antisense cRNA fragment E-F1 of the receptor protein (a), or with ³⁵S-labeled sense control cRNA b). The rat tissue was hybridized with the equivalent rat-specific antisense cRNA probe (c,d). The hybridizing transcripts were visualized as white grains on dark-field reflectance micrographs.

FIG. 10

Immunohistochemical staining of frozen tissue sections derived from

a. human epididymis

b. human epididymis

c. Louse epididymis

d. mouse epididymis

e. bovine epididymis, and

f. bovine epididymis.

In FIGS. 10a, c and e and anti-B antiserum was employed

In FIGS. 10b, d and f pre-immune serum was employed (negative control).

### EXAMPLES

### Example

### RNA Preparation

Total RNA was extracted by the method of J. M. Chirgwin et al., Isolation of biologically active ribonucleic acid from sources enriched in ribonuclease, Biochemistry, 18,5294, (1979), using guanidine isothiocyanate, from frozen tissues of the human testis and epididymis which had been obtained by orchiectomy from men aged 53 and 74 years suffering from prostate carcinoma, and was then purified by caesium chloride density gradient centrifugation.

Using an oligo(dT)-cellulose column, poly(A) RNA could be enriched by affinity chromatography, as described by H. Aviv & Leder, Purification of biologically active globin messenger RNA chromatography on oligothymidylic acid-cellulose, Proc. Natl. Acad. Sci., USA, 69, 1408-1412, (1972). The RNA samples were precipitated with ethanol and, after resuspension in sterile water, stored in a concentration 1 :g/:l at −80 EC.

### Example 2

### In Vitro Translation and Characterization of the Products by Two-Dimensional Gel Electrophoresis

0.5 to 1 :g of the poly(A) RNA of the epididymis and testis from example 1 was in each case translated in vitro in a cell-free system of a reticulocyte lysate from the rabbit (New England Nuclear (NEN), Dreieich, FRG). The synthesis was carried out in the presence of (³⁵S)-methionine (specific activity >1,000 Ci/mol). The resulting protein products were then separated electrophoretically in a two-dimensional gel (cf. P. H. O'Farrel, High resolution two-dimensional electrophoresis of proteins, J. Biol. Chem., 250, 4007-4021 (1975)). For the first dimension, an amount of 350,000 cpm of the (³⁵S)-methionine-labelled translation batches was in each case applied to the gel and focused isoelectrically at an applied voltage of 10,000 V.h. The pH gradient was between 4.0 and 7.5. The separation of the polypeptides according to their molecular weight was carried out in the second dimension within a linear gradient of 7.5-15% acrylamide (cf. D. M. Neville & H. Glossmann, Molecular weight determination of membrane protein and glycoprotein subunits by discontinuous gel electrophoresis in dodecylsulphate, Meth. Enzyme., 32, 92-102, (1974)). To estimate the molecular weights, a ¹⁴C-labeled protein marker (Amersham, GB) was applied in the second dimension. The gel was then subjected to fluorography with the aid of Kodak X-AR5 autoradiographic film (cf. W. M. Bonner & R. A. Laskey, A film detection method for tritium-labeled proteins and nucleic acids in polyacrylamide gels, Europ. J. Biochem., 46, 83-88, (1974)) The exposure time was 8 days. The results are shown in FIG. 2.

FIG. 2, which shows the results, shows the pattern of the translation products derived from epididymis- or testis-specific poly(A) RNA and synthesized in a cell-free system.

In particular, the figure shows

2a: epididymal tissue of a 74 year-old orchiectomy patient treated with cyproterone acetate,

2b: epididymal tissue of a 58 year-old orchiectomy patient not treated with medicaments,

2c: testis tissue of the same patient not treated with medicaments (see FIG. 2b).

Epididymis-specific translation products of which the corresponding bands do not appear in FIG. 2c (translation products of mRNA from the testis) are identified by small arrowheads (see FIG. 2b).

A comparison of FIGS. 2a and 2b shows a change in the expression in epididymal tissue due to previous treatment with antiandrogens. Bands which are reduced in FIG. 2a compared with FIG. 2b have been marked by large arrowheads. The molecular weights of the protein marker are given in kilodalton. Actin(A)- and tubulin(T)-like products are identified in each case.

### Example 3

### Compiling the cDNA Complete Library and Selection of Potentially Specific Clones

The bacteria and bacteriophages were cultured, handled and obtained and the DNA recombinant techniques were used as described by T. Maniatis et al., Molecular Cloning, A Laboratory Manual, Cold Spring Harbour Laboratory, Cold Spring Harbour, N.Y. (1982).

a) Poly(A) RNA from the epididymal tissue of a patient who had not been treated with medicaments was used for compiling the cDNA library (with regard to the pattern of the in vitro translation products, cf. FIG. 2b). 20 :g poly(A) RNA according to example 1 were subjected to reverse transcription using oligo(dT) as a primer (cf. Gubler & B. J. Hoffman., A simple and very efficient method for generating cDNA libraries, Gene, 15, 253-269, (1983)).

The double-stranded cDNA constructs were ligated on both sides with EcoRI-linkers and then inserted into the EcoRI cloning site of the bacteriophage lambda gt11 (Clontech California).

To discover clones of complete length, another epididymis-specific cDNA library was compiled substantially by the method described, but with the deviation that the cDNA constructs were cloned unidirectionally. This was effected by ligating the particular 5′ end with an EcoRI-linker and the particular 3′ end with a XhoI-linker and then inserting the particular constructs into the bacteriophage lambda Uni-ZAP (Stratagene, La Jolla, Calif.).

Bacteria of E. coli stains Y 1090 an XL1-Blue were cultured toe logarithmic phase, transferred to soft agarose, plated our together with the non-amplified lambda library in a phage density of 500 to 1,000 pfu per Petri dish (15 cm) and incubated for 8 hours at 42 EC or 37 EC.

For the differential screening, the plaques of each dish were subsequently replica-plated on two nitrocellulose filters (Schleicher & Schull, Darmstadt, FRG, BA85), the incubation time for the first filter being 1 minute and that for the second filter being 2 minutes.

Replica filters of a total of 20 Petri dishes were or prepared in this manner and then hybridized (positive/negative) with in each case two single-stranded radioactively labelled cDNA probes (cf. b, below)

b) The probes of poly(A) RNA from the epididymis (gives a positive probe) and poly(A) RNA from the testis (gives a negative probe) were prepared as follows using oligo(dT) as the primer:

The two RNA species originated from the corresponding tissues of a patient who had not been exposed to prior treatment with medicaments. In each case 1 :g poly(A) RNA according to example 1 was denatured for 5 minutes at 65 EC in a volume of 2 :l. After rapid cooling of the batches on ice, in each case the following constituents were added in succession:

2 :l oligo(dT) (100 :g/ml)

1 :l dNT-Mix (DATP, dGTP, dTTP, in each case 2 nM)

1 :l 40 mM sodium pyrophosphate

1 :l RNasin (Amersham, GB)

2 :l 10.times. reverse transcriptase buffer (500 mM Tris-Cl (pH 8.5), 500 mM KCl, 100 mM MgCl₂, 100 :g/ml BSA, 10 mM EDTA, 10 mM dithiothreitol

20 units AMV reverse transcriptase (Boehringer Mannheim, FRG)

10 :l (″³²P)-dCTP (NEN, 10 :Ci/:l; S.A.>3,000 Ci/mmol).

The reaction batches were incubated for 15 minutes at 42 EC. After addition of 1 :l “Chasemix” (containing all four dNTPs in a concentration of 10 mM each, the incubation was continued for a further 20 minutes. The reaction was stopped by addition of in each case 1 :l 0.5 M EDTA and the products were precipitated with ethanol without further purification. The probes, marked with a specific activity of >10.sup.8 cpm/:g, were hybridized in parallel with the abovementioned replica filters. The hybridization was carried out in 5.times. Denhardt's solution, 4.times.SET (200 mM Tris (pH 8.0), 20 mM EDTA, 0.6 M NaCl), 0.1% sodium pyrophosphate and 25 mM sodium phosphate buffer (pH 7.0) for 72 hours at 65 EC and a concentration of the radioactivity 5.times.10.sup.6 cpm/ml. The filters were then washed in 0.1% SDS, 2.times. SSC (300 mM sodium chloride, 30 mM sodium citrate) at a temperature at 65 EC.

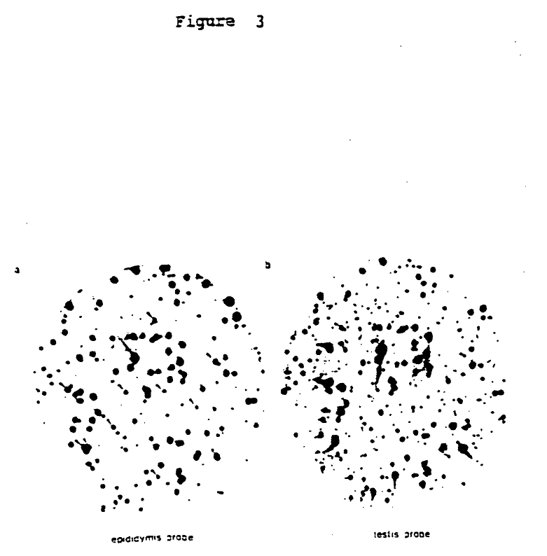

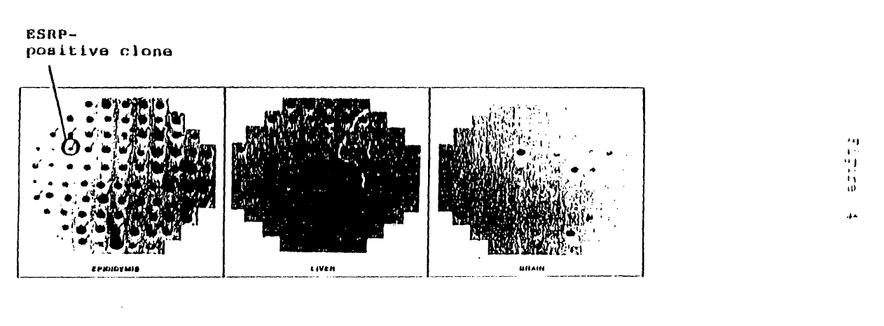

FIG. 3 shows the autoradiograms of two replica filters from one of the Petri dishes which were hybridized with epididymal (a) or testicular b) cDNA probes. The autoradiograms were developed after an exposure time of 16 hours using an amplifying film. The filters represent about 500 independent clones or the cDNA total library form epididymal tissue compiled in the lambda gt11 system. The positive epididymal-specific hybridization signals are marked with arrowheads (cf. FIG. 3a).

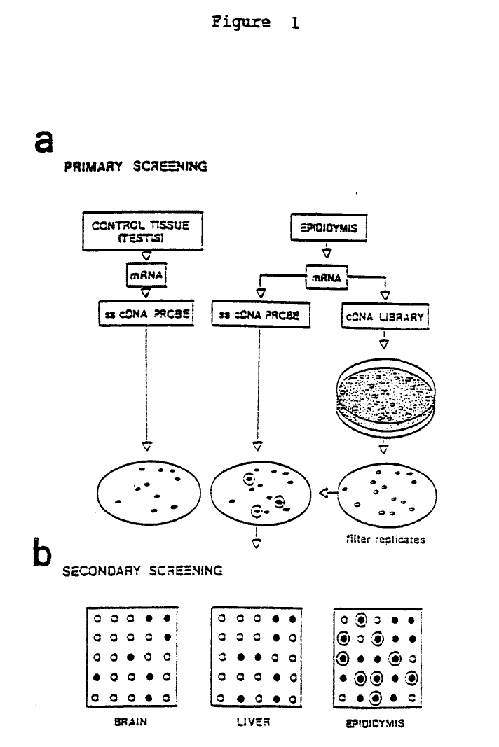

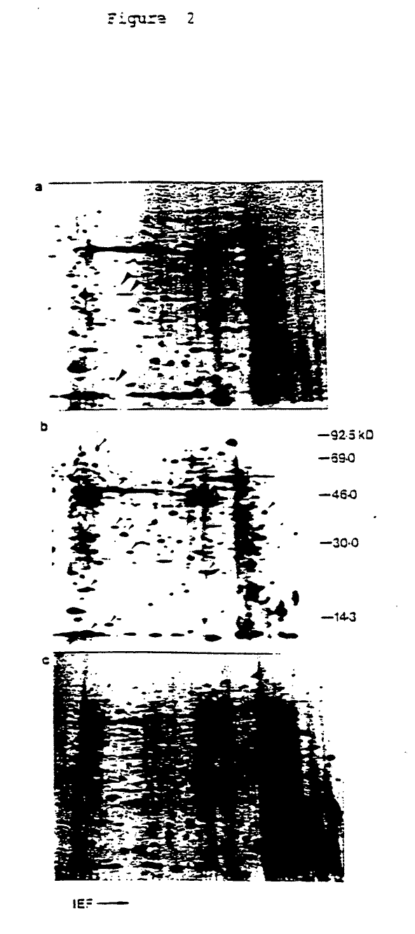

With the aid of this primary screening method (cf. FIG. 1a), it was possible to analyse about 10,000 independent cDNA clones. 265 recombinants of which the signals after hybridization with the epididymal cDNA probe were very much more intense compared with those prepared from testicular tissue were identified. Based on the phage library analysed by screening, the number positive cDNA corresponds to a proportion of 2.5%.

Positive cDNA clones were isolated and divided into groups of up to 80 clones for secondary screening.

The process described above was repeated, with the deviation that in each case 3 replica filters were prepared from each of the Petri dishes covered by groups of in each case 80 clones. To prepare the negative cDNA probes, poly(A).sup.-RNA from the human brain or from the human liver (Clontech California, USA) was used here.

From the analysis of this secondary screening process (cf. FIG. 1b), the number positive recombinants was reduced to 99 clones (cf. FIG. 4).

Positive clones were isolated without further purification of the plaques. The purified lambda DNA was cut with EcoRI and the inserts were isolated by Biotrap elution (Schleicher & Schhll). The EcoRI inserts were subcloned into the bacterial plasmid vector pBS (Stratagene, California, USA.) and inserted by the CaCl.sub.2 transformation method into the bacteria cells of the E. coli stain XL1-Blue (Stratagene, California) (cf. T. Maniatis et al., Molecular Cloning: A Laboratory Manual, Cold Spring Harbour Laboratory, Cold Sprig Harbour, N.Y., (1982)).

### Example 4

### Preparation of Northern Blots for Analysis of the Tissue-Specific Gene Expression and Classification of the Epididymis-Specific cDNA Clones in Families with Related Sequences

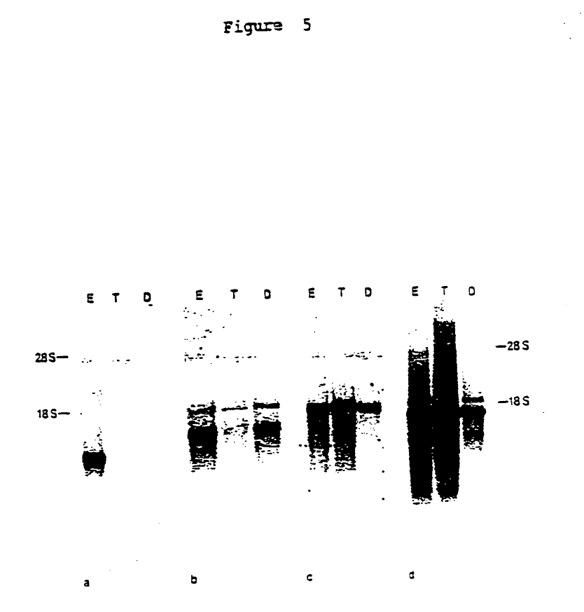

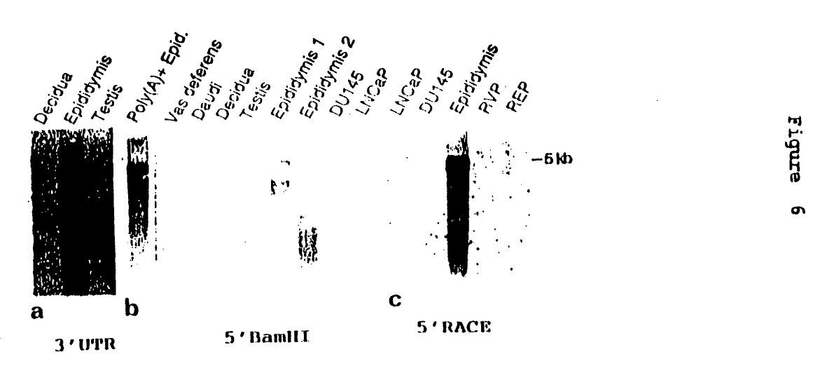

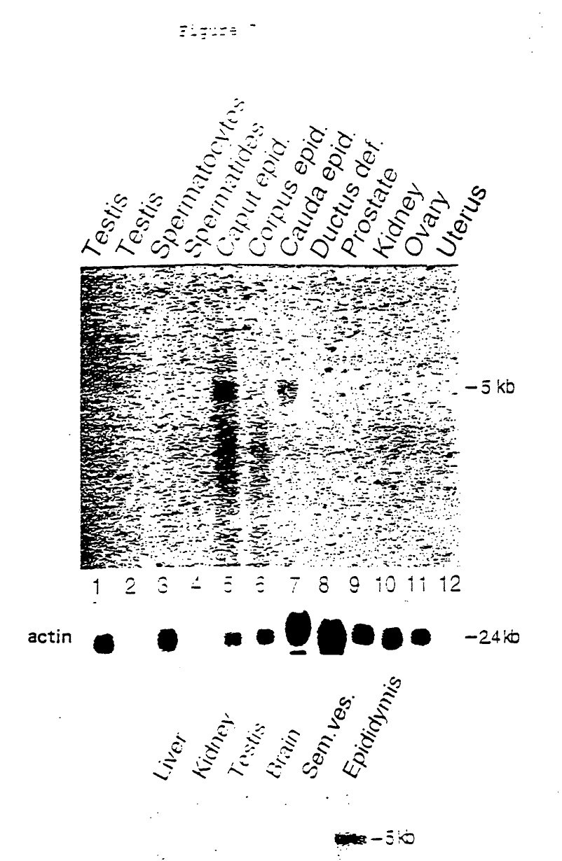

20 :g total RNA from the human epididymis (E) of a patient treated with cyproterone acetate and the same amount of total RNA from the human testis (T) and human decidua (D) were separated electrophoretically in a horizontal 1.3% formaldehyde-agarose gel for 16 hours at a constant voltage of 25 volt (cf. Maniatis et al., Molecular Cloning: A Laboratory Manual, Cold Spring Harbour Laboratory, Cold Spring Harbour, N.Y., (1982) (FIGS. 5,6 and 7)). To determine the size of the RNAs, the gel was additionally loaded with an RNA marker (0.24-9.5 kB). The RNA was then transferred to nylon membranes (Hybond M, Amersham) by capillary blotting in the presence of 20.times.SSC. The blots were subsequently hybridized successively with radioactively potentially epididymis-specific cDNA probes. The probes were isolated by EcoRI restriction of the lambda gt11 DNA subcloned in the plasmid pBS and subsequent Biotrap elution, and after radioactive labeling had a specific radioactivity of >10.sup.9 cpm/:g (cf. A. P. Fainberg and B. Vogelstein, A technique for radiolabeling DNA restriction endonuclease fragments to high specific activity, Analytical Biochemistry, 132, 6-13, (1983). The hybridization was carried out overnight at a radioactivity concentration of 1-2.times.10⁶ cpm/ml. The filters were subsequently washed stepwise with an increasing stringency, starting with 2.times.SSC at room temperature up to 0.1.times.SSC at a temperature of 65 EC. For dehybridization, the filters were incubated for 15 minutes at 65 EC the presence of 2 mM Tris (pH 7.5), 1 mM EDTA and 0.1% SDS. The filters were analysed autoradiographically for the presence of probe material, before they were used for a repeat hybridization with another probe if signals were absent.

FIG. 5 shows examples of the autoradiograms of the northern blot analyses of potentially positive cDNA clones. The exposure time was 20 hours. The clones were evaluated qualitatively in respect of their tissue specificity from the various hybridization patterns.

a) Positive cDNA clone, indicated by epididymis-specific hybridization.

b) Positive but not epididymis-specific hybridization pattern.

c) Positive cDNA clone (*), which also hybridites with rRNA.

d) cDNA clone without tissue-specific expression.

A number of 36 epididymal cDNA clones could be identified with the aid of this method. The specific hybridization signal of each clone insert with epididymal RNA was at least one order of magnitude more intense than that with RNA from the testis or decidua (cf. FIG. 5a).

Using the results of cross-hybridizations the original phage library, the 36 epididymis-specific cDNA clones could be assigned to six independent families, each of which comprised a set of clones related to one another. It was found that five of the cDNA families are derived from different, relatively short mRNA, molecules having an average length of 600 to 1,000 nucleotides, while the clone according to the invention, which contains the genetic information for the present receptor protein, has a length of about 5,000 nucleotides.

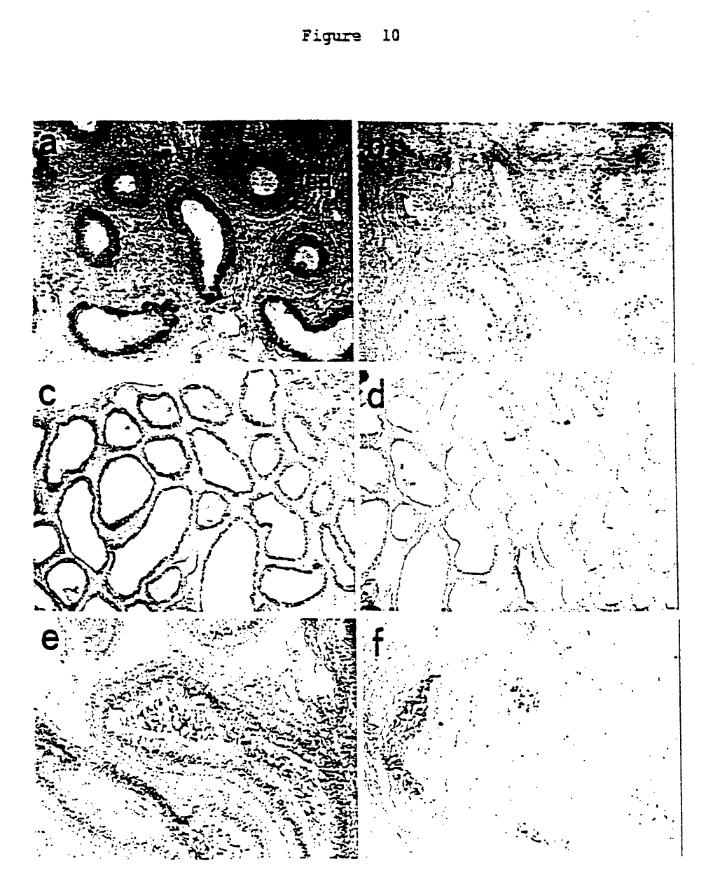

Since only cDNA fragments having a maximum length of 3.7 kb could also be isolated from the second cDNA library (cf. FIG. 8b), but on the basis of the results of the northern hybridization an mRNA length of about 5 kb was to be assumed, the 5′ start of this cDNA was cloned using the 5′ RACE method (RACE=rapid amplification of cDNA ends; M. A. Frohman et al., “Rapid production of full-length cDNAs from rare transcripts: Amplification using a single gene-specific oligonucleotide primer”, Proc. Nad. Acad. Sci. USA, 85, p. 8998-9002 (1988)). For this, two gene-specific antisense primers A1 and A2 were prepared from the 5′ region of the known cDNA sequence comprising 3.7 kb (A1=5′AGC TAT GGG AGC TGA AG 3′ (SEQ ID NO:9) A2=5′TGT CAA TGG CAG GGG TG 3′ (SEQ ID NO: 9)). With the aid of these primers, starting from 1 :g total RNA from the human epididymis, a 5′ fragment having a length of 996 nucleotides, which overlapped with the already known cDNA sequence over a length of 49 nucleotides (incl. A1 primer) was obtained using the protocol “5′ RACE System for Rapid Amplification of cDNA Ends” from Gibco BRL, Berlin (cf. FIG. 9b, 5′ RACE-fragment, 1.0 kb).

### Example 5

### Checking of the Tissue Specificity of the Clone According to the Invention

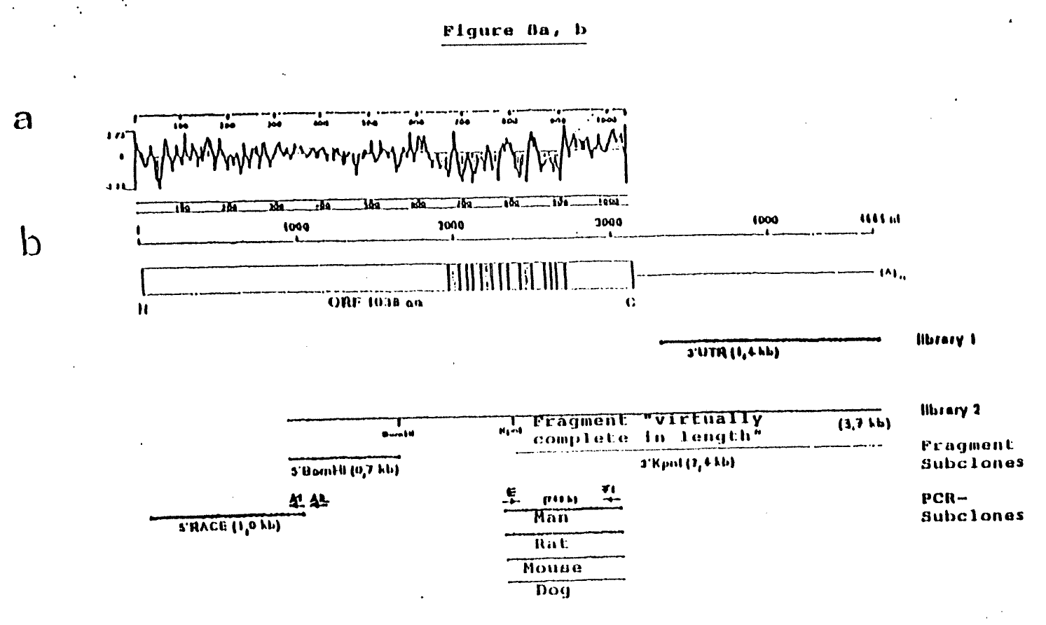

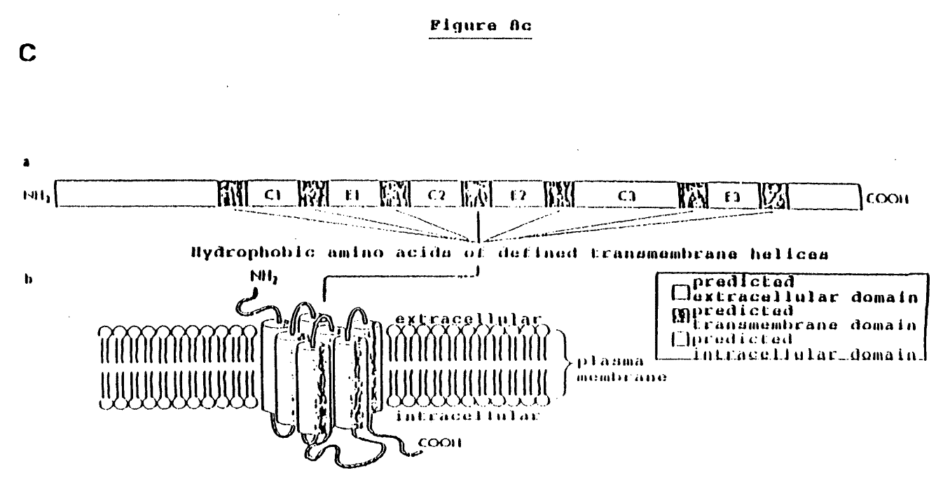

In each case 20 :g total N RNA the human decidua, epididymis and tests were separated and transferred to nylon membranes according to example 4. To check the tissue specificity of the clone according to the invention, in each case 10 :g poly(A) RNA from the epididymis and total RNA from the seminal duct (vas deferens), and in each case 20 :g total RNA from a lymphoblastoid cell line (Daudi), from prostate carcinoma cell lines (LNCaP, DU145) and from embryonal, epididymal cell lines (RVP, REP) were also separated analogously and likewise applied to a nylon membrane. A ³²P-labelled cDNA insert from the 3′ region of the clone according the invention (cf. FIG. 8b, 3′ UTR) was employed as the probe in FIG. 6a, and was hybridized with northern blots according to example 4. In FIG. 6b, in contrast, a ³²P-labelled BamHI cDNA insert from the 5′ region of the clone according to the invention was employed (cf. FIG. 8b, 5′ BamHI fragment). FIG. 6c shows a northern blot after hybridization with a ³²P-labelled 5′ RACE fragment of the clone according to the invention.

The results shown in FIG. 6a-c show that the cDNA clone according to the invention gives an unambiguous hybridization signal or only with the epididymal RNA. Furthermore, with the aid of the autoradiogram corresponding to FIG. 6c, the genuineness of the 5′ RACE fragment could be confirmed (note the exclusive signal with epididymal RNA at approx. 5 kb). No cross-hybridization was to be detected in other types of human tissue analyzed (16 in total).

### Example 6

### Detection of Homologous Sequences in the Epididymis of Other Species of Mammals and Checking of the Tissue Specificity of the Clone According to the Invention in the Rat

To investigate whether homologues of the receptor protein according to the invention are also expressed with the same tissue specificity in other species of mammals an attempt was made to amplify corresponding fragments from epididymal total RNA from the rat, mouse and dog with the human subfragment E-F1 (cf. FIG. 3b, PCR subclones, prepared using the two primers E=5′CAT CCG AAA ATA CAT CC 3′ (SEQ ID NO:10) and F1=5′TGA AGG CAC ACA TCT CC 3′ (SEQ ID NO:11)) using the PCR technique (gen. ref cf. R. S. Cha and W. G. Thilly, Specificity, Efficiency and Fidelity of, PCR Methods and Applications, vol. 3, p. 18-29 (1993)).

In each case 5 :g total RNA from the animal species mentioned were subjected to annealing in a total volume of in each case 20 :l using 0.5 :g oligo(dT)₁₂₋₁₈ (Pharmacia, Freiburg). The subsequent first strand synthesis was carried out in the presence of in each case 10 mM DATP, cGTP, dGTP and dTTP and of 200 units of reverse transcriptase (Superscript, Gibco BRL, Berlin). For the subsequent PCR amplification, in each case 1 :l of the products of the first strand synthesis were mixed with in each case 10 mM of the four dNTPs, in each case 20.mu.M of the human gene-specific-primers E and F1 (see above) and with in each case 7 units of Taq-Polymerase (Promega, Heidelberg) in a total volume of in each case 50 :l and subjected to the following PCR cycles: denaturing (5 minutes at 95 EC); then 30 cycles comprising denaturing (1 minute at 95 EC), annealing (1 minute at 55 EC) and elongation (1 minute at 72 EC). The complementary cDNA fragments obtained in this manner were separated electrophoretically in 1.2% agarose gels. Fragments having a length of about 750 nucleotides were then electroeluted and inserted into the cloning vector pCR11 (Invitrogen, ITC Biotechnology, Heidelberg). The sequence of the cloned DNA fragments was determined as described in the following example 7 and showed—both a the nucleotide and at the amino acid level—a particular homology of about 90% to the human sequence. This surprising result shows that the DNA sequence which codes for the receptor protein according to the invention has a high degree of conservation at least among mammals.

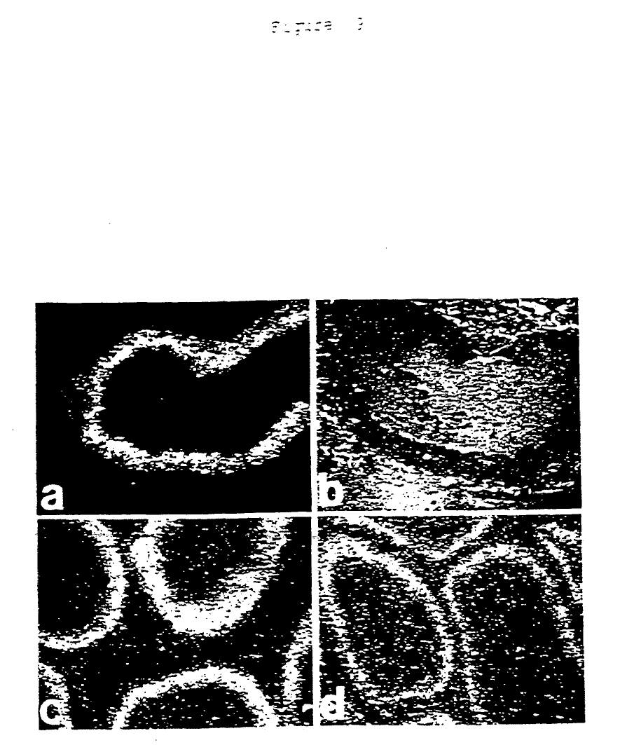

For control of the tissue specificity of the clone according to the invention in the rat, in each case 20 :g of total RNA from various rat tissues (with the exception of the 2nd lane in FIG. 7 (testis), only 4 :g) were separated and transferred to nylon membranes according to example 4. A ³²P-labelled cDNA insert (spec. activity 19.sup.9 cpm/:g) from the rat homologue of the clone according to the invention (E-F1) was employed as the probe and hybridized with northern blots according to example 4. The autoradiograms of the northern blot analysis for the clone according to the invention developed after an exposure time of 15 hours are shown in diagram form in FIG. 7. A rehybridization of the blot shown above with a control actin probe is shown in the middle of FIG. 7, which shows that approximately equimolar amounts of RNA were found in all the lanes of the gel.

FIG. 7 shows that probe E-F1 from the rat hybridized exclusively with epididymal RNA, the signals being the most intense within the epididymis in the caput proximal region) and in the cauda (distal region) (cf. lanes 5-7).

The results in the rat confirm the epididymis-specific expression pattern of the sequence according to the invention, since no cross-hybridization was to be detected in the other types of tissue analysed (20 in total).

### Example 7

### Determination of the Nucleotide Sequence and Derived Amino Acid Sequence of the cDNA Clone According to the Invention

The base sequence of the longest cDNA clone according to the invention of examples 4, 5 and 6 was determined with the aid of the dideoxy method of Sanger, F. and Coulson, A. R. “A rapid method for determining sequences in DNA by primed synthesis with DNA polymerase” J. Mol. Biol. 94, 441 (1975). For this purpose, subclones were prepared according to example 3 and converted into single-stranded DNAs under alkaline conditions.

The result for the clone according to the invention is shown in SEQ ID NO: 1.

FIG. 8a shows the result of the hydrophobicity plot, hydrophobic amino acid sequences being shown below the zero line. The hydrophobic amino acid position 620 and position 900 of the amino acid sequence which characterize the 7 transmembrane domains and lead to the conclusion of a receptor having the structure shown generally in FIG. 8c (from J. D. Watson et al., (1993)) are striking. The extracellular N-terminus comprising 620 amino acids is furthermore noteworthy. A corresponding bar diagram of the protein according to the invention is shown in FIG. 8b. It shows the open reading frame (ORF) in the form box having a length of 3,114 nucleotides, which corresponds to a coding capacity of 1,038 amino acids (aa). The 7 transmembrane domains are emphasized by shading and corresponding to the results of the hydrophobicity analysis. The nucleotide sequence from position 3,115 to position 4,665 includes the 3′ untranslated region (3′ UTR) of the clone according to the invention and approximately corresponds to the cDNA isolated from the first cDNA library. The two cDNA clones isolated and sequenced from the two cDNA libraries of the epididymis (library 1/library 2) are shown below the bar diagram. Finally, PCR subclones and the primers employed for their particular synthesis (A1, A2, E, F1) are shown underneath. The fragments used as probes are emphasized as bold lines.

### Example 8

### In Situ Transcript Hybridization of the mRNA According to the Invention in Epididymal Tissue Sections from Man and the Rat

A specific probe was prepared by in vitro transcription using the 743 bp long fragment E-F1 (cf. ex. 6) of the DNA sequence according to the invention in the presence of [″³⁵S]CTP and was employed for determination of the expression of the sequence according to the invention in frozen sections.

The results of corresponding in situ hybridization experiments are shown in FIG. 9. The white dots of the frozen sections of human origin shown in figure a using a dark-field microscope show specific signals from the in situ hybridization in the presence of the above fragment as an “antisense” probe. Figure b represents the result of a control experiment carried out with a comparable tissue section, in which instead of the “antisense” probe used previously, the complementary strand was used as a “sense” probe (negative control).

Figure a clearly shows that the DNA sequences according to the invention is expressed exclusively in the epithelial layer of the epididymis, but not in the stroma cells or muscle cells of the same human tissue.

Figures c and d show experiments carried out in parallel using tissue sections of the rat epididymis. Figure c shows a section of the proximal region of the epididymis, while in diagram d the distal region of the same tissue is shown. Comparing diagrams c and d, it becomes clear that the proximal region produces more intense signals, which is probably to be attributed to a functional specification this region.

### Example 9

### Preparation of Antigens Via Chemical Synthesis for Induction of Antibodies and Use of Obtained Antibodies in Immunohistochemistry

Several sequence motifs within SEQ ID NO: 1 were identified by computer (DNA-Star Star software package Promega, Madison, Wis., USA) as possible antigenic epitopes of the receptor protein according to the invention and synthesized chemically, together with flanking amino acids by the method of Merrifield J. M. Stewart, “Synthesis ard use of neuropeptides”, Neuroendocrine Peptide Methodology, ed. P. M. Conn, Academic Press, New York, p. 815-844 (1989)) (Table 1).

1TABLE I List of oligopeptides of the receptor protein used as antigens for immunization (numbers corresponding to codons of SEQ ID No 1) Codon length of localisation within the Antigen No. oligopeptide receptor protein N1 141-154 14 aa.sup.1) N-terminal (SEQ ID NO: 3) N2 266-280 15 aa N-terminal (SEQ ID NO: 4) A 785-799 15 aa second extracellular loop (SEQ ID NO: 5) within 7TM²) domain B 799-808 10 aa second extracellular loop (SEQ ID NO: 6) within 7TM domain C 834-848 15 aa third intracellular loop (SEQ ID NO: 7) within 7TM domain .sup.1)aa=amino acids²)7TM=seven transmembrane

The immunogenic polypeptides N1, N2, A, B, and C according to the invention prepared in the manner described before were each coupled to carrier substances (keyhole limpet haemocyanin, KLH) using m-maleimidobenzoic acid N-hydroxysuccinamide ester (M8759, Sigma (Sambrook et al., Molecular Cloning—A Laboratory Manual, 2nd edition, Cold Spring Harbor Laboratory Press (1989)). Those resulting compounds were employed as antigens for immunization of rabbits, chickens and rats to generate antibodies.

Antisera obtained from rats after immunisation with A, B, and C immunogens, respectively, were employed for the determination of the expression of the receptor protein according to the invention in frozen sections of humans, mouse and bovine epididymis. Detection was performed by known immunohistological methods using a commercially available secondary biotinylated rabbit anti-rat antibody at a dilution of 1:100 (Dako E 468, Hamburg, Germany) (Harlow and Lane, Antibodies—A Laboratory Manual, Cold Spring Harbour Laboratory Press, Cold Spring Harbour, New York (1988)). The total procedure was carried out at room temperature. 10 :m cryosections were fixed in 4% paraformaldehyde for 10 min. After three washes in PBS (phosphate buffered saline pH 7.2) the sections were blocked in 5% normal rabbit serum in PBS. First antibody was applied for 1 h. After three washes in PBS the secondary antibody was applied for 30 min. After additional three washes in PBS, a streptavidin-biotin-horserad-dish peroxidase complex (K0377, Dako, Hamburg, Germany) was applied according to Sternberger et al., Immunocytochemistry, J. Wiley and Sons, New York (1979). Subsequently, the staining was carried out by incubation (Sternberger) with the chromogen diaminobenzidine (Dako, C 3467).

The results of the immunohistochemical experiments are shown in FIG. 10. In FIGS. 10a and b results of experiments are shown which were carried out with tissue derived from human epididymis. The dark staining of epithelial (ep) cells of human origin shown in FIG. 10a was achieved using anti-B antiserum (dilution 1:600). FIG. 10b shows the result of a control experiment carried out with a comparable tissue section, in which instead of the anti-B antiserum, the preimmune serum of the same animal was used (negative control).

FIG. 10a clearly shows that the protein epitope recognized by anti-B-antiserum according to the invention is present exclusively in the epithelial layer of the epididymis, but not in stroma cells or muscle cells or within the lumen of the epididymal duct of the same human tissue.

FIGS. 10c and d show experiments carried out in parallel using tissue sections of mouse epididymis.

FIGS. 10e and f show experiments carried out in parallel using tissue sections of bovine epididymis.

FIGS. 10c and e the anti-B-antiserum was employed, whereas in FIGS. 10d and f the pre-immune serum was employed (negative controls).

The same results were obtained using anti-A and anti-C antisera (data not shown).

The results of this example demonstrate the following:

1. The use of three different antisera against different epitopes of the receptor protein, namely anti-A, anti-B or anti-C antisera, resulted in identical epithelial cell staining. This confirms the presence of the receptor protein according to the invention within the epithelium of the epididymis.

2. The receptor protein according to the invention is expressed in a cell type specific manner.

3. The high degree of homology of the receptor protein at least among mammalian species is confirmed, as the antisera against the human receptor also recognize homologues in mouse and bull.

4. The fact that antisera against A, B, and C epitopes are also reacting with mouse and bull confirm the hypothesis that the receptor protein according the invention is a “principal” structural molecule of epididymal epithelium.

The results of this example further demonstrate the extremely high cell type specificity of the receptor protein according to the invention and accordingly the suitability of the substances proposed according to the invention for specific manipulation of the functions of the epididymis in the maturation of spermatozoa, Furthermore it can be concluded that the sequences according to the invention, including fragments thereof, can be used as specific probes for both qualitative and quantitative diagnostic investigations of biopsy samples. Moreover, the extremely high expression rate of the receptor protein according to the invention indicates its suitability as a promising mediator the context of therapeutic or contraceptive purposes.

### Example 10

### Use of the Receptor Protein According to the Invention and of the cDNA Coding for this for Isolating Specific Ligands

To isolate specific ligands for the receptor protein according to the invention, the N-terminal extracellular domain (from position 1 to 620 of the amino acid sequence according to SEQ ID NO: 2) of the receptor protein was prepared in a eukaryotic expression system such as the cell lines COS-7, HEK 393 and MDCK. For this purpose, the cDNA region which codes for this domain (corresponding to position 1 to 1,860 of the nucleotide sequence according to SEQ ID NO: 1) was provided on the 3′ end with a flag sequence, i.e. an oligonucleotide sequence, which codes for a known, highly specific peptide epitope, and cloned into the expression vector pRc/CMV and pTracer-CMV (Invitrogen, San Diego, Calif., USA). After transfection of the cell lines with the expression vector described, the fusion product was obtained by known processes and purified by affinity chromatography using immobilized antibodies directed against the flag epitope.

The fusion product was then employed as probe in a conventional protein screening process (cf. J. Sambrook, E. F. Fritsch and T. Maniatis, Molecular Cloning: A Laboratory Manual, Cold Spring Harbor Laboratory, Cold Spring Harbor, N.Y., chapter 2 (1989)) using a cDNA expression library from the human testis prepared in a lambda bacteriophage. For this, about 1 million independent cDNA bacterial clones which express testicular products and therefore potential ligands by IPTG induction were transferred to nylon membrane filters and incubated with the recombinant receptor-binding domain under specifically were then removed under stringent washing conditions, so that only specific binding complexes which could be rendered visual via the flag epitope with conventional antibody detection systems (system using alkaline phosphate, Sigma, Deisanhofen) were present on the filter. The phage colonies identified in this manner were isolated and purified and subjected to a sequence determination.

In order to ascertain whether the ligands discovered can induce a signal transduction by the receptor according to the invention and are therefore suitable for simulating maturation of spermatozoa in subfertile mammals, cultures of the cell lines mentioned above transfected with the total cDNA construct with and without a flag epitope on the 3′ end were incubated separately with the positive ligands, and their change in the intracellular cAMP level and/or the Ca.sup.2+ content caused by the specific binding was determined. Positive ligands which were not capable of causing such changes are suitable, for example, as antagonists for inhibition of maturation of spermatozoa and can be employed for the preparation of contraceptive agents.

The use of such ligands for therapeutic purposes is particularly advantageous, since as a result of the tissue-specific expression of the receptor protein according to the invention, this type of tissue is influenced selectively and no side effects, or only minor side effects, are therefore to be expected.

The documents cited herein are incorporated in their entirety herein by references.

1. An isolated mammalian epididymis-specific receptor protein which has the amino acid sequence shown in SEQ ID NO: 2, or a derivative of said protein or a fragment of said protein, said derivative or fragment having at least one biological activity and/or immunogenicity of said protein, wherein said derivative or fragment comprises at least ten contiguous amino acids of SEQ ID NO: 2.

2. A protein of claim 1 wherein said derivative or fragment comprises a hydrophilic region of said receptor.

3. A protein of claim 2 wherein said derivative or fragment comprises an extracellular hydrophilic region of said receptor.

4. An isolated protein having a sequence selected from the group consisting of SEQ ID NOs: 2, 3, 4, 5, 6 and 7.

5. A protein of claim 1 herein said derivative or fragment is comprises at least one sequence selected from the group consisting of any one of SEQ ID NO: 3-7.

6. An isolated DNA sequence which codes for the receptor protein or an active derivative or fragment thereof having the same biological activity and/or immunogenicity, according to claim 1.

7. An isolated DNA sequence which codes for a protein of claim 3.

8. An isolated DNA sequence which codes for a protein of claim 4.

9. An isolated DNA sequence according to claim 6, chosen from a) the nucleotide sequence shown in SEQ ID NO: 1, b) the sequence of nucleotides 1 to 3,114 of SEQ ID NO: 1, c) a sequence homologous to the sequence represented by SEQ ID NO: 1 having a degree of homology of at least 70% and d) a syngenic or complementary sequence of a sequence according to a), b) or c), or a fragment thereof, where said sequence codes for a protein or polypeptide having the same biological activity and/or immunogenicity as said protein or active derivative or fragment.

10. A vector molecule, comprising at least one of the DNA sequence according to claim 2 as an insert, while maintaining the ability to replicate in a suitable host cell.

11. A vector molecule according to claim 10, wherein said DNA sequence is inserted in said vector, in a manner such that expression thereof can take place in a suitable host organism.

12. A prokaryotic or eukaryotic host cell transformed with a vector molecule according to claim 10.

13. A prokaryotic or eukaryotic host cell transformed with a vector molecule according to claim 11.

14. A process for the preparation of an isolated mammalian epididymis-specific receptor protein, which has an amino acid shown in SEQ ID NO: 2 or a derivative or fragment thereof having at least one biological activity and/or immunogenicity of said protein, said process comprising culturing a host cell according to claim 12 in a culture batch under conditions which allow expression of the DNA sequence, and obtaining the expression product from the culture batch.

15. An isolated antibody, which reacts with and is specific to at least one epitope included in a protein or polypeptide according to claim 1.

16. The antibody of claim 15 wherein said antibody is a monoclonal antibody.

17. A pharmaceutical composition which comprises a protein, derivative or fragment according to claim 1 as an active component.

18. A pharmaceutical composition which comprises at least one antibody according to claim 15 as an active component.

19. A pharmaceutical composition which comprises, as an active component, at least one nucleotide sequence which hybridizes with a nucleotide sequence according to claim 6.

20. A pharmaceutical composition according to claim 19, further comprising a detectable marker.

21. A composition comprising a protein according to claim 4.

22. A pharmaceutical composition according to claim 17 for treatment of male reproduction disorders or for contraception.

23. A method of isolating a ligand specific for an epididymis-specific receptor comprising incubating the epididymis-specific receptor protein of claim 1 with a substance suspected to be a ligand of said receptor and detecting binding of said receptor to said ligand.

24. A method according to claim 23 wherein said ligand is an agonist of said epididymis-specific receptor.

25. A method according claim 23 wherein said ligand is an antagonist of said epididymis-specific receptor.

26. A method of treating infertility in a male mammal comprising administering an agonist of an epididymis-specific receptor protein of claim 1 to said made mammal.

27. A contraceptive method for male mammals comprising administering an antagonist of an epididymis-specific receptor to said male mammal wherein said antagonist comprises a protein derivative or fragment of claim 1.

28. A method of treating infertility in a male mammal comprising administering an agonist of an epididymis-specific receptor of claim 1 to said male mammal.

29. A contraceptive method for male mammals comprising administering an antagonist of an epididymis-specific receptor of claim 1 to said male mammal.

30. A method of diagnosing infertility in a male comprising measuring from said male to an epididymis-specific receptor protein of claim 1.
